(19) United States
(12) Patent Application Publication
(10) Pub. No.: US 2023/0412962 A1
(43) Pub. Date: Dec. 21, 2023

(54) HEADPHONE APPARATUS

(71) Applicant: KOKOON TECHNOLOGY LIMITED, London (GB)

(72) Inventors: Guillaume PARRIN, Horsham Sussex (GB); Richard HALL, Royston Hertfordshire (GB); Timothy ANTOS, London (GB)

(21) Appl. No.: 18/246,871

(22) PCT Filed: Sep. 28, 2021
(86) PCT No.: PCT/GB2021/052514

§ 371 Date: Mar. 28, 2023

(30) Foreign Application Priority Data
Sep. 28, 2020 (GB) 2015349.0

(51) Int. Cl. H04R 1/10; A61B 5/024

(52) U.S. Cl. CPC H04R 1/1033; H04R 1/105; A61B 5/02416; A61B 5/02438; H04R 2420/07; H04R 1/1016

(57) ABSTRACT

Headphone apparatus including earpieces and a module including an audio signal source. The earpieces are each tethered to the module by elongate elastic connectors that each include a wire for carrying the electrical audio signals from the module to the earpiece. Each elongate elastic connector has a preformed geometry such that a first part of each elongate elastic connector provides an ear hook that, when worn, lies directly over the otobasion superius of the wearer, and a second part of each elongate elastic connector provides a tension spring that provides a tension on the ear hook when the apparatus is worn.

### CROSS-REFERENCE TO RELATED APPLICATIONS

This application claims priority from and is related to the following prior application Patent Cooperation Treaty Patent PCT/GB2021/052514, filed on Sep. 28, 2021, which claims priority from Great Britain Patent Application No. 2015349.0, filed on Sep. 28, 2020. These prior applications, including the entirety of their written description and drawings, are collectively hereby incorporated by reference into the present application.

### BACKGROUND

The present invention relates to headphone apparatus of a type comprising in-the-ear earphones in which the earpieces are adapted to be fitted directly in the outer ears of a wearer.

For headphones specially intended to be worn in bed and/or while the wearer is sleeping, it is desirable the headphones are as comfortable as possible when worn lying down in order that they do not interfere with the wearer's sleep or ability to fall asleep.

An advantage of headphones that comprise in-ear earpieces, as opposed to over-ear earphones, is that they offer the potential for greater comfort when the wearer is in a lateral recumbent position. However, there is a tendency for the earpieces to become dislodged from the ear. This commonly arises when a cable connected to the earpiece becomes pinched between the body and a surface on which the wearer is lying and as a result becomes overly taut, pulling on the earpiece when the wearer moves.

A solution is to incorporate the cable into a relatively rigid guide piece that extends over the ear. The guide piece retains the earpiece against the ear and acts to shield the earpiece from tension on the cable that would otherwise dislodge the earpiece. However, this solution renders the headphones uncomfortable to wear in a lateral recumbent position due to the thickness of the rigid guide piece transferring pressure from a pillow (or other surface on which the wearer's head is rested) to the side of the skull or between the ear helix and the side of the skull.

Wireless ear-buds incorporate a wireless transceiver within each earpiece and as result do not include a cable. However, their greater size—a consequence of their need to house a wireless transceiver and a battery—also make them uncomfortable to wear in a lateral recumbent position. In addition, they are typically only able to operate for a few hours before needing to be recharged. This makes them unsuitable for applications that require continuous operation for many hours.

CN206506645 relates to headphone apparatus comprises ear phones tethered together by a wave shaped connector having a steel wire inner core 21 and a plastic covering. The connector is malleable meaning it can be bent and deformed to enable a user to change the clamping curvature of the headset according to match their head size (paragraph 27). A variant version shown in FIGS. 4 & 5 includes an electronic module 24 and an audio wire runs alongside the steel wire.

The headphones of the present invention were devised to improve upon those of the prior art.

### BRIEF SUMMARY

According to an aspect of the invention there is provided headphone apparatus comprising: earphones adapted to be fitted directly in the outer ears of a wearer; a module comprising an audio signal source; and separate elongate connectors extending between the module and the respective earphones to carry audio signals received by the wireless receiver to the earphones; wherein each elongate connector comprises a resiliently flexible electrical cable to carry the audio signals and, a portion of each elongate connector has a pre-formed shape to provide an ear hook, which when worn, extends directly over an ear of the wearer; and characterized in that a part of the ear hook that, when worn, lies directly over the otobasion superius has a flexibility that is substantially the same as the flexibility of the resiliently flexible electrical cable.

The portion of the ear hook adapted to sit directly over the otobasion superius may thus consist of the pre-form shaped electrical cable, i.e. without an added structural stiffener element. In its simplest form the ear hook and optionally the whole elongate connector may consist of the electrical cable.

By omitting the stiffening structures that sit over the wearer's ears, the headphones are more comfortable to wear when worn in a laterally recumbent position. Additionally the more flexible ear hook allows the elongate connector's effective length to increase more readily in response to an applied tension on the cable, e.g. as a result of the wearer turning their head. This reduces the strain placed on the user's ear making the headphones apparatus more comfortable to wear. Further, the increased flexibility allows for the pre-formed electrical cable to conform more readily to the shape of the head of the wearer making it less likely to slip off over the ear.

Favorably, when the apparatus is worn, each elongate connector is relatively taut with a slight tension applied to the ear hook. In this way the elongate connectors are caused to lie more tightly against the body which helps to retain the ear hook over the ear. Additionally, it reduces the likelihood of an elongate connector becoming pinched between the wearer's body and a surface they are lying on in such a way that would result in an excessive tension on the earphone.

A desirable degree of tension can be applied to the ear hook by incorporating a buckle or other fastener type element allowing the length of the elongate connector to be adjusted. Alternatively, the elongate connector may include a tension spring for applying a constant tension force to the ear hook. At least one of the resiliently flexible electrical cables may include a spring portion, the electrical cable within the spring portion having a geometry to define the tension spring.

In addition to providing a constant tension, the spring has the effect of providing a degree of resilient flexibility to the elongate connector about its longitudinal axis. The spring allows the effective length of the elongate cable to vary in response to movement of the wearer's head relative to their body, e.g. when they turn their head to the side.

The spring portion of the cable may have a serpentine geometry. In contrast to a helical spring, a serpentine geometry allows for a spring with a relatively flat (planer) form which can more comfortably seat against the head and/or neck of the wearer, especially when the side of the wearer's head is rested against a surface, e.g. when lying in bed. A disadvantage of a serpentine geometry is that the spring tends to be stiffer compared with a helical geometry, however this is compensated for by the relatively flexible ear hook.

The headphone apparatus may be adapted, e.g. the length of the elongate cables selected, such that when worn, the module is retained against the back of the user's head and/or neck. An external side of the module may be concavely curved to conform with a curve of the wearer's head and/or neck in the transverse plane.

The elongate connectors may be rigidly connected to the module at points lying on a transverse plane that also includes the module's center of gravity.

At least one of the earphones may comprise a body sensor oriented, when worn, such that a sensing aperture of the body sensor, e.g. including a photoplethysmography (PPG) sensor, faces a cavum concha of the wearer's ear; the body sensor adapted to outputs signals indicative of a physiological variable (e.g. the heart-rate) of the wearer; and wherein the module comprises a transmitter to wirelessly transmit the output signals received from the sensor via the electrical cable. Through integration of the body sensor, the headphone apparatus can be used with an audio-for-sleep system to select and/or generate audio files to play to the wearer based on a perceived condition and/or state inferred from the measured physiological variable(s).

The elongate connector may have a preformed geometry in which: serpentine bends of the tension spring lie in a plane; a portion of the ear hook proximal to the spring, which when the headphone apparatus worn lies posterior to the ear, extends out of the plane in a first direction; and a second portion of the ear hook distal from the spring, and which when the headphone apparatus is worn lies in front of the ear, extends from the terminus of the first portion in an opposition direction back through the plane.

Through this geometry the elongate connector can more closely conform with the curvature of the head without impinging with the front of the helix of the ear, making the headphones more comfortable to wear and less likely to slip off.

To provide an ear hook of a size sufficient to extend around ears of most sizes, the ear hook may have a radius of curvature equal or greater than 12 mm.

### BRIEF DESCRIPTION OF THE DRAWINGS

The accompanying figures in which like reference numerals refer to identical or functionally similar elements throughout the separate views, and which together with the detailed description below are incorporated in and form part of the specification, serve to further illustrate various embodiments and to explain various principles and advantages all in accordance with the present disclosure, in which:

FIG. 1 is a perspective view of headphone apparatus;

FIG. 2 is a perspective view of headphone apparatus from the opposite side

FIG. 3A illustrate the headphone apparatus worn by a user with small ears and a small ear-to-ear distance;

FIG. 3B illustrates the headphone apparatus worn by a user with large ears and a large ear-to-ear distance;

FIG. 4 illustrates the module showing the connection between the module and the elongate connectors;

FIGS. 5A and 5B are side views of the cable illustrating it at rest and stretched;

FIG. 6 is a top view of the cable showing the pre-formed bending of the hook out of the plan of the spring;

FIG. 7 is a front view of the cable;

FIG. 8A illustrates the earphone and ear hook worn by a wearer with a small ear;

FIG. 8B illustrates the earphone and ear hook worn by a wearer with a large ear;

FIG. 8C illustrates the earphone and ear hook worn by a wearer with a very large ear;

FIG. 9A illustrates the earphone and ear hook worn by a wearer with a small ear-to-ear distance;

FIG. 9B illustrates the earphone and ear hook worn by a wearer with a large ear-to-ear distance; and

FIGS. 10 and 11 illustrate variant geometries of the cable.

### DETAILED DESCRIPTION

As required, detailed embodiments are disclosed herein; however, it is to be understood that the disclosed embodiments are merely examples and that the devices and methods described herein can be embodied in various forms. Therefore, specific structural and functional details disclosed herein are not to be interpreted as limiting, but merely as a basis for the claims and as a representative basis for teaching one of ordinary skill in the art to variously employ the disclosed subject matter in virtually any appropriately detailed structure and function. Further, the terms and phrases used herein are not intended to be limiting, but rather, to provide an understandable description. Additionally, unless otherwise specifically expressed or clearly understood from the context of use, a term as used herein describes the singular and/or the plural of that term.

The terms “a” or “an”, as used herein, are defined as one or more than one. The term “plurality”, as used herein, is defined as two or more than two. The term “another”, as used herein, is defined as at least a second or more. The terms “including” and “having,” as used herein, are defined as comprising i.e., open language. The term “coupled,” as used herein, is defined as “connected,” although not necessarily directly, and not necessarily mechanically.

It will also be understood that when an element is referred to as being “connected” or “coupled” to another element, it can be directly connected or coupled to the other element or intervening elements can be present. In contrast, when an element is referred to as being “directly connected” or “directly coupled” to another element, there are no intervening elements present.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

The term ear-to-ear distance used herein refers to a distance between the entrance of the ear canals via the back of the head.

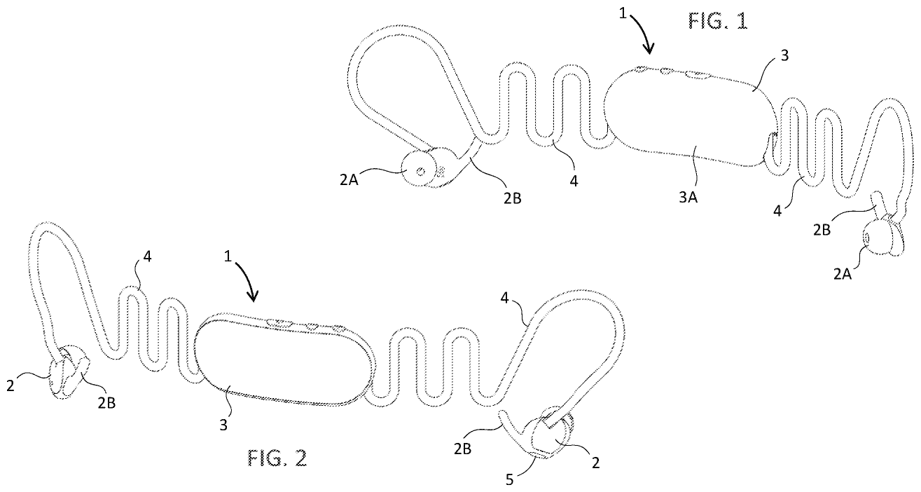

With reference to FIGS. 1 and 2 there is shown headphone apparatus 1 adapted to be worn around the head of a wearer. The apparatus 1 comprises earphones 2 shaped to sit in the wearer's outer ears and a module 3 comprising a wireless transceiver. Each earphone 2 is communicatively coupled to the module 3 by separate elongate connectors provided by separate electrical cables 4.

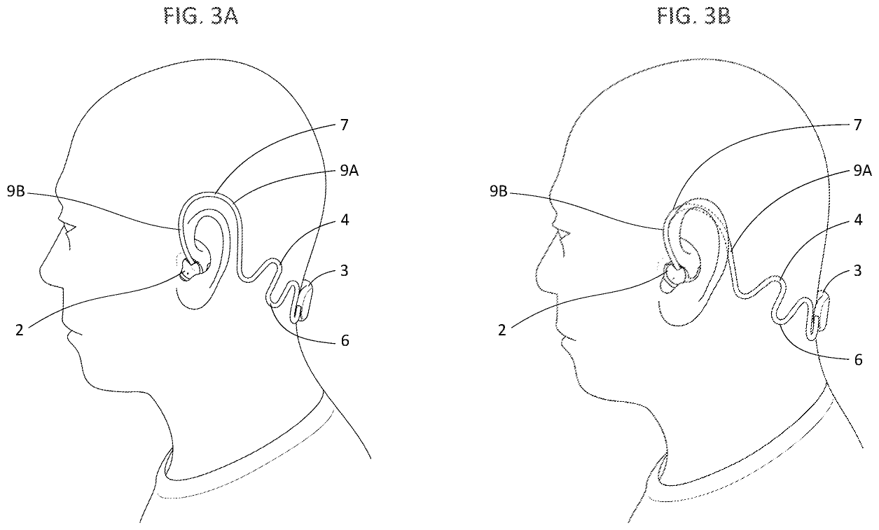

As can be seen in FIGS. 3A and 3B, when worn, the module 3 sits towards the base of the back of the wearer's head and/or the top of the neck extending across the medial sagittal plane. Each electrical cable 4 extends from the module 3 around the respective left and/or right of the head, over and around the top of the ear in front of the root of helix before dropping down to follow a path between helix crux and the tragus, against the anterior notch and connecting to the earphone 2.

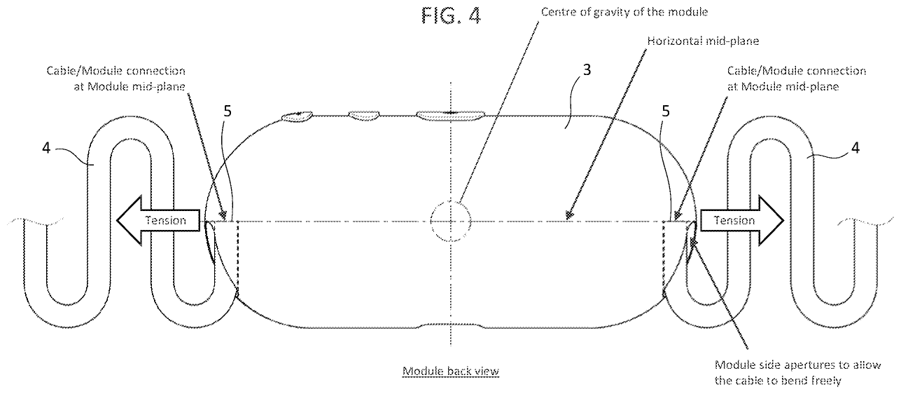

With reference to FIG. 4, each cable 4 is rigidly supported (e.g. clamped) to the module 3 about points 5 that lie between the top and bottom edges of the module 3 body, ideally at or close to the mid-horizontal plane. This keeps the module 3 balanced and flush to back of the wearer's head and/or neck. As best seen in FIGS. 1 and 3, an outer face of the module 3 that lies directly against the wearer defines a concave surface 3A in order to approximately conform with the curve of the head and/or neck in the transverse plane.

Referring back to FIGS. 1 and 2, the body of each earphone 2 comprises an ear tip 2A and a tail 2B. A loudspeaker sits within the earphone ear tip 2A to convert an electrical audio signal received from the wireless module 3 through the cable 4, into sound which is carried out through the ear tip 2 into the ear canal. The tail 2B is adapted to sit against the posterior concha to stabilize the earphone 2 within the ear.

One or both of the earphones 2 also comprise a photoplethysmography (PPG) sensor to infer the heart rate of the wearer. The PPG sensor has a sensing aperture 5 provided in a bottom surface of the earphone 2 that, when the earphone 2 is worn, faces and is preferably in contact with the cavum concha.

The module 3 is adapted to receive wireless audio signals from a remote device (not shown), e.g. a smart phone paired with the headphone apparatus, and to relay the output signals (PPG signals) from the PPG sensor, and/or information derived from processing the PPG signals, to the remote device.

The remote device is adapted to run a software program that uses a determined heart rate (and/or variable derived from the heart rate e.g. heart rate variability) derived from the output of the PPG sensor, to infer a state or condition of the wearer—e.g. whether the wearer has fallen asleep or stage within a sleep pattern, and to select and/or generate one or more audio files to be transmitted to the headphone apparatus 1 in order to be played to the wearer.

Additionally, one or each earphone 2 includes a microphone (not shown). The microphone may be used to provide a voice call function as is well known. Alternatively or additionally it/they may be used to detect ambient noise that provides an input to an active notice cancellation circuitry housed in the module 3. An output audio signal of the active notice cancellation is mixed with the audio signal received by the transceiver module 3 before being transmitted along the cables 4 to the earphones 2

Each cable 4 is comprised of an electrically conductive core, e.g. one or more copper wires, and a sleeve of electrically insulative synthetic (e.g. plastic) material. In one example the sleeve comprises an inner sleeve of a thermoplastic elastomer (TPE) and an outer, co-axial, sleeve of a thermoplastic polyurethane (TPU). In another example both the inner sleeve and outer sleeve comprise TPU.

The composition and thickness of the sleeves are chosen such that each cable 4 has the property of being resiliently flexible when deformed in directions normal to the cable's longitudinal axis. The metal core of the cable 4 means the cable 4 is relatively non-elastic in cable's longitudinal axis. The formation of such cables is well known in the art.

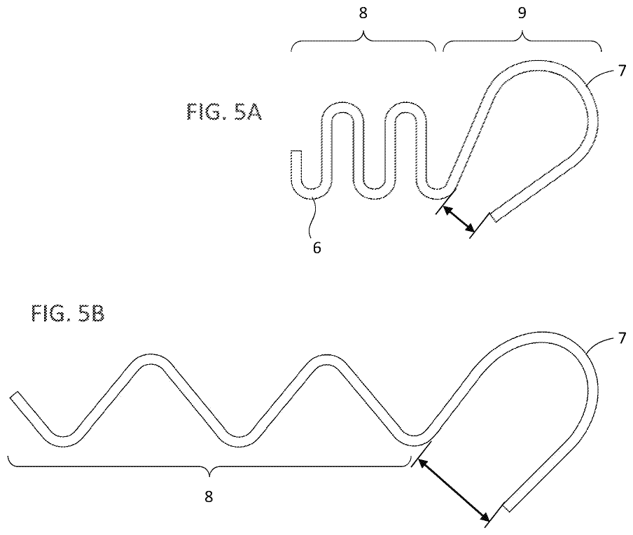

With reference to FIG. 5A, each cable 4 is manufactured (e.g. molded) into a pre-formed shape defining a number of curves to provide a geometry that defines a spring 6 and an ear hook 7. As the cable 4 is resiliently flexible, the cable 4 will revert towards its preformed shape and size at rest, i.e. in the absence of an applied external force.

A spring portion 8 of the cable 4 defines a series of curves forming the serpentine tension spring 6. The spring 6 allows the effective length of the elongate connector between the earphone 2 and the module 3 to vary in order to compensate for different ear-to-ear distances. It also keeps the electrical cable 4, including the ear hook 7, under tension and against the wearer's body.

An ear hook portion 9 of the electrical cable 4 extending between the spring portion 8 and the earphone 2, defines a curve with radius of curvature great enough to enable the cable 4, when worn, to extend over the otobasion superius, and sit at a position anterior to the pinna of the ear. To achieve this, the radius of curvature is greater or equal to 12 mm. In contrast, the radius of curvature of the curves of the serpentine may be less than 12 mm to provide a sufficient number of loops that the spring 6 can stretch to accommodate wearers with large ear to ear distances.

FIG. 5B illustrates the electrical cable 4 highly extended as would be the case if worn by a user having a very large ear-to-ear distance and large ears. The extended spring 6 and ear loop 7 both increase the overall effective length of the elongate connector.

The sinuosities of the serpentine spring 6 are formed so that the cable 4 lies flat against the wearer's head and/or neck when worn, i.e. the sinuosities of the cable 4 to form the spring 6 trace a path that lies on a surface approximating the surface of the wear's head and/or neck. This makes the cable 4 more comfortable to wear compared, for example, with a helix spring configuration.

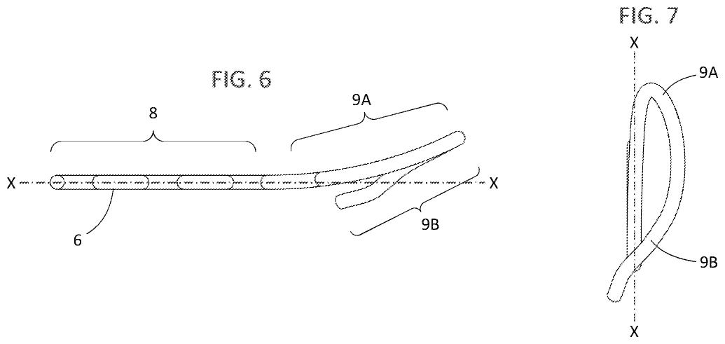

In one embodiment, the sinuosities are pre-formed in a single plane as shown in FIGS. 6 and 7. A first section 9A of the cable 4 of the ear hook portion 9, which extends from the terminus of the spring portion 8, is shaped to extend out of the plane (represented by dashed line X-X) of the loops of the spring 6 in a direction so as to conform with the curve of the skull in the transverse plane of the wearer, above the ear. A second section 9B of the cable 4 within the ear hook portion 9 extends from the terminus of the first section 9A to the earphone 2. The second section 9B extends in an opposite direction to the first section 9A, back towards and through the plane of the spring 6 in order to follow a path between the helix crux and tragus. Where the sinuosities of the spring 6 are pre-formed to lie on a curved surface, the first section 9A of the ear hook may be formed so as to extend away from the spring portion 8 at a different angle compared with the angles between the legs of the cable that form the sinuosities of the spring. Expressed another way, there the ear hook 7 may extend away from the spring at an angle disjointed from the curves that form the spring.

FIGS. 3A and 3B illustrate how the combination of the spring 6 and the relative high flexibility of the ear hook 7 deform under tension depending on the ear size and ear-to-ear distance of the wearer.

FIG. 3A illustrates the headphone apparatus 1 worn by a person having relatively small ears and a small ear-to-ear distance. The top of the ear hook 7 lies above the top of the helix superior against the side of the wearer's head. The relatively short length of the elongate connector at rest means that the spring 6 is slightly stretched (cf. FIG. 5A) when worn in order to provide a constant low tension force on the rear end of the ear hook 7 ensuring that the cable 4 of the ear hook 7 remains flush against the head.

The tension on the ear hook 7 translates to a rotational force on the earphone 2 with the axis of rotation extending through the ear tip 2A of the earphone 2. The rotational force tends to rotate the earphone 1 such that the bottom surface of the earphone 2, in which the sensing aperture 5 of the PPG sensor is located, makes good contact with the cavum concha. This enables the PPG sensor to take readings more reliably.

FIG. 3B illustrates the headphone apparatus 1 worn by a person having relatively large ears and a large ear-to-ear distance. Both the spring 6 and ear hook 7 are more stretched compared with the configuration of FIG. 3A to compensate for the greater ear-to-ear distance. The curve of the ear hook 7 is opened out, i.e. the cable 4 is straightened, increasing the separation between the two ends of the ear hook, i.e. that connected to the earphone 2 and that connected to the spring 6. The ear hook 7 passes behind the pinna, i.e. between the pinna and the skull, and against the otobasion superius.

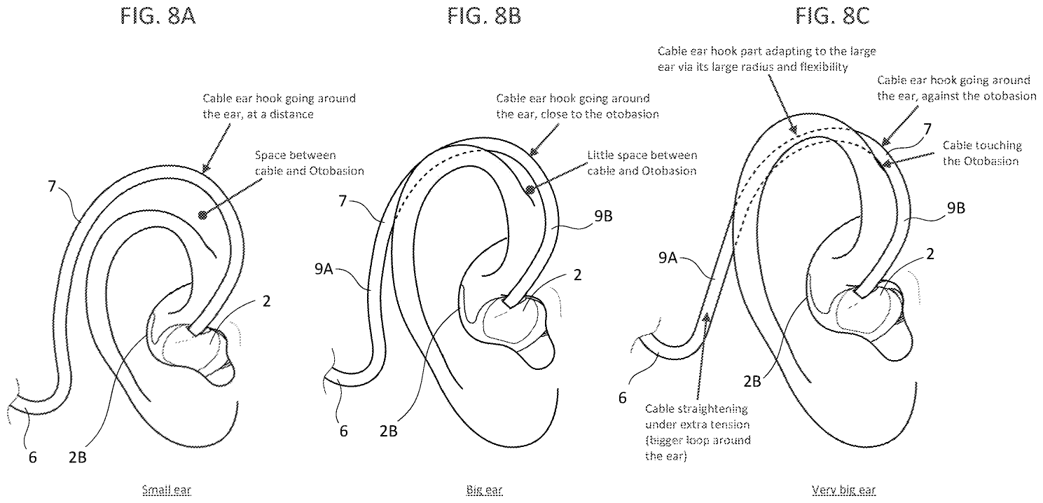

FIG. 8A-8C show in greater detail how the flexible ear hook 7 adapts to small, large, and very large ears. The large radius of curvature of the ear hook 7 allows it to sit around the ear irrespective of the ear size ensuring that the second section 9A of the ear hook 7 lies to the anterior of the pinna before tracing a path over the otobasion. With smaller ears, the ear hook 7 lies above the pinna whereas with larger ears the cable 4 passes closer and may contact the otobasion passing directly between the pinna and the skull.

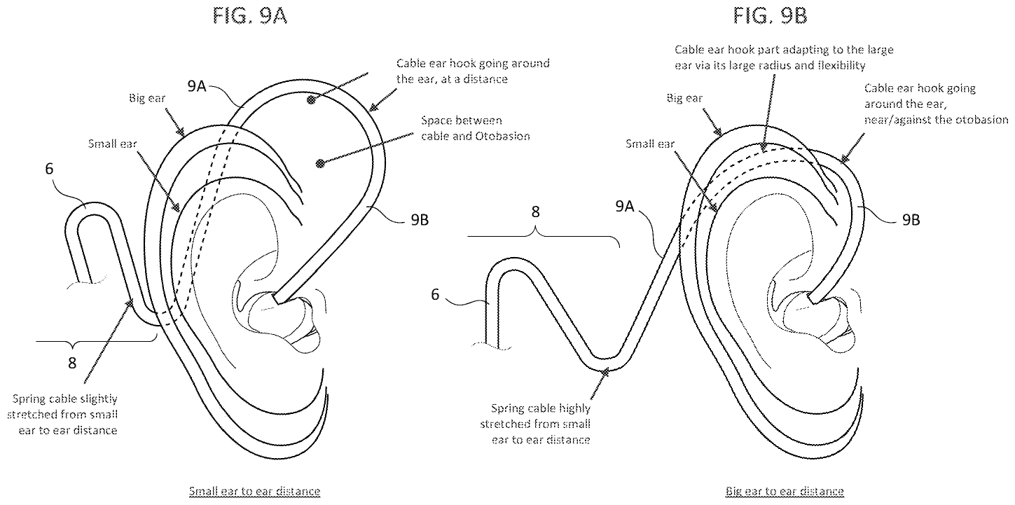

FIGS. 9A and 9B illustrate in greater detail how the flexible ear hook 7 stretches to conform with larger ear-to-ear distance. As can be seen in FIG. 9A, the end of the ear hook 7 that connects with the spring 6 lies relatively far forward, proximate to the posterior of the ear. The loop of the ear hook 7 also extends forward above and anterior to the ear forming a gap between the cable 4 and the otobasion. With a large ear-to-ear distance, the ear hook 7 is elongated such that the end of the ear hook 7 that connects to the spring 6 sits further away from the posterior of the ear. The curve of the hook 7 is drawn against the otobasion and passes between the pinna and the skull.

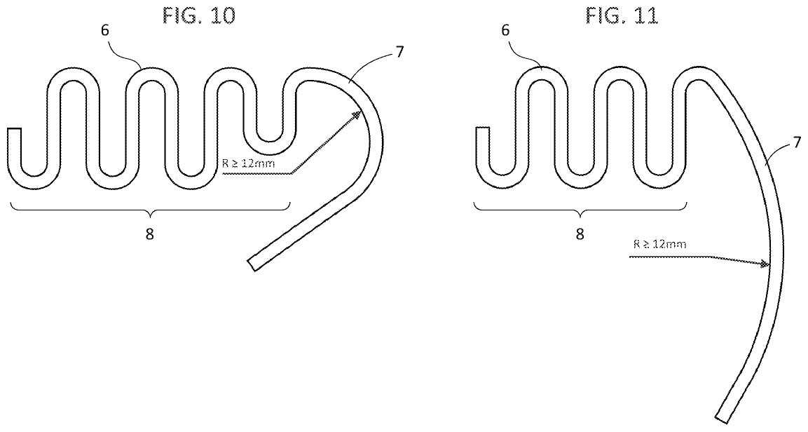

FIGS. 10 and 11 illustrate variant geometries of cable 4 to provide different shapes of ear hook 7. In each case the ear hook 7 comprises a curve or loop with a radius of curvature equal or greater than 12 mm.

Although less desirable, a variant embodiment may omit the spring 6 and rely on a buckle fastener or equivalent to allow for the length of the elongate connectors to be adjusted.

The headphone apparatus 1 may comprise a body sensor other than or in addition to the photoplethysmography sensor to derive heart rate or another physiological variable. Examples include EEG, EDR and sensors to measure body temperature. The headphone apparatus may also include one or more sensors to detect movement of the wearer and or environmental conditions. In a more simple variant of the headphone apparatus 1, the body sensor may be omitted.

The module 3 may be adapted to be sited elsewhere to the back of the head/neck, e.g. entirely on one side of the medial sagittal plane.

Instead or in addition to including a wireless transceiver, the module 3 may include a computer readable store for holding audio files to be played to the wearer. Where no wireless receiver is present, the module may comprise an I/O port, e.g. USB for receiving audio files from an external device through a wired connection.

The Abstract is provided with the understanding that it is not intended be used to interpret or limit the scope or meaning of the claims. In addition, in the foregoing Detailed Description, various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

The corresponding structures, materials, acts, and equivalents of all means or step plus function elements in the claims below are intended to include any structure, material, or act for performing the function in combination with other claimed elements as specifically claimed. The description herein has been presented for purposes of illustration and description, but is not intended to be exhaustive or limited to the examples in the form disclosed. Many modifications and variations will be apparent to those of ordinary skill in the art without departing from the scope of the examples presented or claimed. The disclosed embodiments were chosen and described in order to explain the principles of the embodiments and the practical application, and to enable others of ordinary skill in the art to understand the various embodiments with various modifications as are suited to the particular use contemplated. It is intended that the appended claims below cover any and all such applications, modifications, and variations within the scope of the embodiments.

Although specific embodiments of the subject matter have been disclosed, those having ordinary skill in the art will understand that changes can be made to the specific embodiments without departing from the scope of the disclosed subject matter. The scope of the disclosure is not to be restricted, therefore, to the specific embodiments, and it is intended that the appended claims cover any and all such applications, modifications, and embodiments within the scope of the present disclosure.

What is claimed is:

1. A headphone apparatus comprising:
earphones adapted to be fitted directly in the outer ears of a wearer;
a module comprising an audio signal source;
and separate elongate connectors extending between the module and the respective earphones to carry audio signals from the audio signal source to the earphones;
wherein each elongate connector comprises a resiliently flexible electrical cable to carry the audio signals and, a portion of each elongate connector has a pre-formed shape to provide an ear hook, which when worn, extends directly over an ear of the wearer; and
characterized in that a section of each elongate connector that provides a part of the ear hook that, when worn, lies directly over the otobasion superius of the wearer, has a flexibility that is substantially the same as the flexibility of the resiliently flexible electrical cable in directions normal to the longitudinal axis of the elongate connector; and
wherein at least one of the resiliently flexible electrical cable elongate connectors includes a spring portion, which when the headphone apparatus is worn, lies in a position posterior to the ear of the wearer, the electrical cable within the spring portion having a pre-formed geometry to define a tension spring to apply a tension force to the ear hook.

2. The headphone apparatus according to claim wherein the electrical cable within the spring portion has a serpentine geometry.

3. The headphone apparatus according to claim wherein, when worn, the bends of the serpentine lie on a surface that corresponds with the surface of the head and/or neck of the wearer.

4. The headphone apparatus according to claim wherein at least one of the earphones comprises a body sensor oriented, when worn, such that a sensing aperture of the body sensor faces a cavum concha of the wearer's ear; the body sensor adapted to output signals indicative of the heart-rate of the wearer; the module adapted to receive the signals via the electrical cable.

5. The headphone apparatus according to claim 1, wherein the electrical cable within the spring portion has a serpentine geometry, and wherein the elongate connector has a preformed geometry in which;
the serpentine bends of the spring lie on a plane; and
a first portion of the ear hook proximal to the spring, which when the headphone apparatus worn lies behind the ear, extends out of the plane in a first direction; and a second portion of the ear hook distal from the spring, and which when the headphone apparatus is worn lies in front of the ear, extends from the terminus of the first portion in an opposition direction back through the plane.

6. The headphone apparatus according to claim 1, wherein the audio signal source comprises a wireless receiver for receiving wireless audio signals.

7. The headphone apparatus according to claim 1, wherein an external side of the module is concavely curved to conform with a curve of a wearer's head and/or neck in a transverse plane.

8. A headphone apparatus comprising:
earphones adapted to be fitted directly in the outer ears of a wearer;
a module comprising an audio signal source; and
separate elongate connectors extending between the module and the respective earphones to carry audio signals from the audio signal source to the earphones;
wherein each elongate connector comprises a resiliently flexible electrical cable to carry the audio signals and, a portion of each elongate connector has a pre-formed shape to provide an ear hook, which when worn, extends directly over an ear of the wearer; and
wherein a section of each elongate connector that provides a part of the ear hook that, when worn, lies directly over the otobasion superius of the wearer, has a flexibility that is substantially the same as the flexibility of the resiliently flexible electrical cable in directions normal to the longitudinal axis of the elongate connector; and
wherein at least one of the resiliently flexible electrical cable elongate connectors includes a spring portion, which when the headphone apparatus is worn, lies in a position posterior to the ear of the wearer, the electrical cable within the spring portion having a pre-formed geometry to define a tension spring to continuously apply a tension force to the ear hook; and
wherein the tension spring allows the effective length of the elongate connector between the earphone and the module to continuously vary to compensate for different ear-to-ear distances while keeping the resiliently flexible electrical cable under tension and against a wearer's body.

9. The headphone apparatus of claim 8, wherein each of the two resiliently flexible electrical cable elongate connectors includes a spring portion, which when the headphone apparatus is worn, lies in a position posterior to the respective ear of the wearer, the electrical cable within the spring portion having a pre-formed geometry to define a tension spring to continuously apply a tension force to the respective ear hook; and
wherein the tension spring allows the effective length of each of the two elongate connectors between each respective earphone and the module to continuously vary to compensate for different ear-to-ear distances while keeping the resiliently flexible electrical cable under tension and against a wearer's body.
